(19) United States
(12) Patent Application Publication
(10) Pub. No.: US 2026/0263552 A1
(43) Pub. Date: Sep. 10, 2026

(54) PHARMACEUTICAL COMPOSITIONS FOR IMPROVING ORAL BIOAVAILABILITY

(71) Applicant: Bristol-Myers Squibb Company, Princeton, NJ (US)

(72) Inventors: Bo LANG, Somerset, NJ (US); David John GOOD, Westfield, NJ (US); Neil Raymond MATHIAS, North Brunswick, NJ (US); Divyakant S. DESAI, West Windsor, NJ (US); Monika LAVAN, Jersey City, NJ (US); Freddy ARCE, Raritan, NJ (US); Stephen M. CARL, Howell, NJ (US); Olafur S. GUDMUNDSSON, West Windsor, NJ (US); Kimberly Ann FOSTER, Yardley, PA (US)

(21) Appl. No.: 19/166,159

(22) PCT Filed: Mar. 15, 2024
(86) PCT No.: PCT/US2024/020225

§ 371 Date: Sep. 17, 2025

Related U.S. Application Data
(60) Provisional application No. 63/490,929, filed on Mar. 17, 2023.
(60) Provisional application No. 63/498,207, filed on Apr. 25, 2023.

(51) Int. Cl. A61K 38/12; A61K 9/20; A61K 9/28; A61K 31/185

(52) U.S. Cl. CPC A61K 38/12; A61K 9/2009; A61K 9/2018; A61K 9/2054; A61K 9/2853; A61K 31/185

(57) ABSTRACT

In accordance with the present disclosure, pharmaceutical formulations that improve the oral bioavailability of cyclic peptides, have been discovered.

### CROSS-REFERENCE TO RELATED APPLICATIONS

This application claims the priority benefit of U.S. Provisional Application No. 63/490,929, filed Mar. 17, 2023, and U.S. Provisional Application No. 63/498,207, filed Apr. 25, 2023, both of which are incorporated herein by reference in their entireties.

### FIELD

The present disclosure provides pharmaceutical compositions which improve the oral bioavailability of cyclic peptides. Also disclosed are pharmaceutical compositions comprising macrocyclic compounds that bind to PD-L1 and are capable of inhibiting the interaction of PD-L1 with PD-1 and CD80.

### BACKGROUND

Despite an increasing trend in drug discovery favoring larger molecules such as millamolecules, poor oral bioavailability remains an impediment for more widespread use. Oral administration of large, hydrophilic molecules is a challenge due to pH and gastric/small intestinal enzyme interaction and low intestinal epithelial membrane permeability resulting from minimal passive or carrier-mediated transcellular permeation across phospholipid bilayers, as well as restricted paracellular transport through tight junctions. Other variables, such as plasma half-life and therapeutic index also impact the feasibility of the oral delivery of millamolecules.

In recent years, several cyclic peptides that block the interaction of PD-L1 with either PD-1 or CD80 have been reported (see, for example, U.S. Pat. Nos. 9,308,263; 9,850,283; 9,879,046; and 9,856,292). Such compounds are useful for enhancing, stimulating, and/or increasing an immune response in patients and can be used in the treatment of conditions such as septic shock and cancer. While oral delivery of these molecules would offer numerous advantages over delivery through injection, as in the case of other larger-sized molecules, developing oral formulations of such molecules has proven challenging. Thus, there is a need for oral formulations that provide improved bioavailability of larger biologically active molecules such as cyclic peptides.

### SUMMARY

The present disclosure pharmaceutical compositions which improve the oral bioavailability of cyclic peptides.

In a first aspect the present disclosure provides a pharmaceutical composition comprising: a cyclic peptide; and a decanoic acid salt. In some aspects, the decanoic acid salt is sodium decanoate. In some aspects, the cyclic peptide is Compound (I) of the formula:

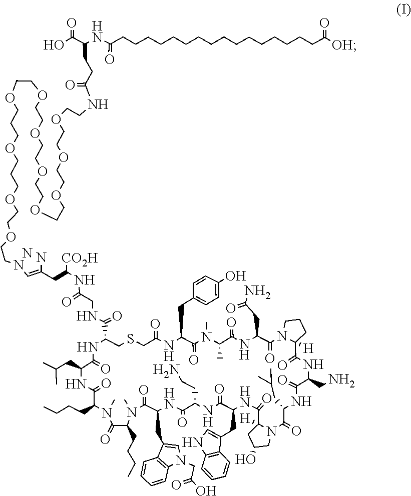 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof.

In certain aspects, the cyclic peptide is present in an amount of about 0.1 wt % to about 20 wt %. In some aspects, the cyclic peptide is present in an amount of about 0.5 wt % to about 10 wt %. In some aspects, the cyclic peptide is present in an amount of about 6.0 wt % to about 9.50 wt %. In some aspects, the cyclic peptide is present in an amount of about 0.8 wt % to about 5.0 wt %.

In certain aspects, the decanoic acid salt is sodium decanoate. In some aspects, the sodium decanoate is present in an amount of about 15 wt % to about 70 wt %. In some aspects, the sodium decanoate is present in an amount of about 20 wt % to about 55 wt %. In some aspects, the sodium decanoate is present in an amount of about 20 wt % to about 30 wt %. In some aspects, the sodium decanoate is present in an amount of about 27 wt % to about 28 wt %.

In certain aspects, the composition is for oral administration. In some aspects, the composition comprises a capsule, tablet, minitablet, or sachet.

In certain aspects, the present disclosure provides a pharmaceutical composition comprising:
a. Compound (I) having the formula:

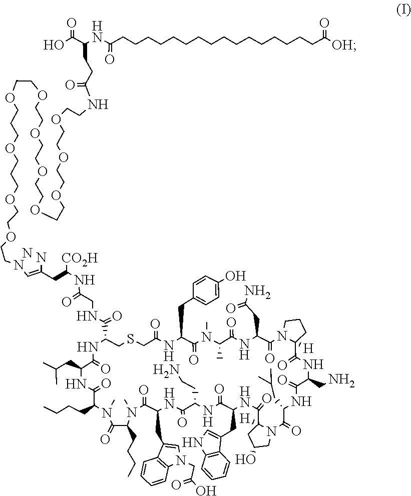 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof,
b. about 27.5 wt % sodium decanoate;
c. about 37 wt % to about 43 wt % microcrystalline cellulose;
d. about 18 wt % to about 22 wt % mannitol;
e. about 4 wt % croscarmellose sodium;
f. about 2 wt % silicon dioxide; and
g. about 2 wt % magnesium stearate.

In some aspects, Compound (I), or the pharmaceutically acceptable salt thereof, is present in an amount of about 0.8%. In some aspects, Compound (I), or the pharmaceutically acceptable salt thereof, is present in an amount of about 1.7%. In some aspects, Compound (I), or the pharmaceutically acceptable salt thereof, is present in an amount of about 3.3%. In some aspects, Compound (I), or the pharmaceutically acceptable salt thereof, is present in an amount of about 6.7%. In some aspects, Compound (I), or the pharmaceutically acceptable salt thereof, is present in an amount of about 8.3%.

In certain aspects, the composition is for oral administration. In some aspects, the composition comprises a capsule, tablet, minitablet, or sachet.

In some aspects, the pharmaceutical composition comprises sodium decanoate wherein the sodium decanoate is crystallized.

In certain aspects, the present disclosure provides a method of improving bioavailability of a cyclic peptide in a subject in need thereof comprising formulating the cyclic peptide with sodium decanoate. In some aspects, the bioavailability is improved at least by about 0.1%, at least by about 0.2%, at least by about 0.3%, at least by about 0.4%, at least by about 0.5%, at least by about 0.6%, at least by about 0.7%, at least by about 0.8%, at least by about 0.9%, or at least about 1.0%. In some aspects, the cyclic peptide is Compound (I) having the formula:

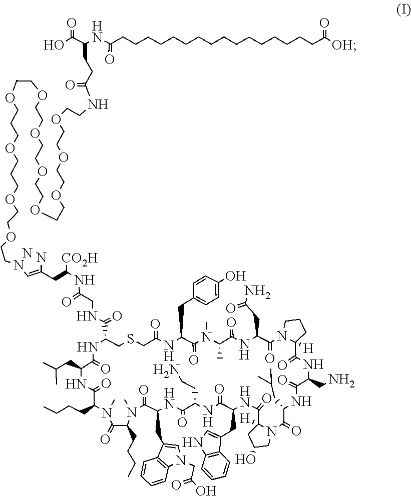 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof. In some aspects, the sodium decanoate is crystallized.

In certain aspects, the present disclosure provides a method of inhibiting growth, proliferation, or metastasis of cancer cells in a subject in need thereof, said method comprising administering to the subject a therapeutically effective amount of a pharmaceutical composition described herein. In some aspects, the cancer is selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and a hematological malignancy.

In certain aspects, the present disclosure provides a method of enhancing, stimulating, and/or increasing an immune response in a subject in need thereof, said method comprising administering to the subject a therapeutically effective amount of a pharmaceutical described herein.

### BRIEF DESCRIPTION OF THE FIGURES

FIG. 1 shows the plasma concentration of Compound (I) over time in cyno monkeys dosed with 12 mg of Compound (I) formulated as described in Example 1.

FIG. 2 shows the plasma concentration of Compound (I) over time in dogs treated with 20 mg of Compound (I) formulated as described in Example 3.

FIG. 3 shows the plasma concentration of Compound (I) over time in dogs treated with 100 mg of Compound (I) formulated as described in Examples 5A & 5B.

FIG. 4 shows the plasma concentration of Compound (I) over time in dogs treated with 100 mg of Compound (I) formulated as described in Examples 5B, 5C, & 5D.

### DETAILED DESCRIPTION

The present disclosure is directed toward a pharmaceutical composition comprising a a cyclic peptide and a decanoic acid salt such as sodium decanoate. The composition can improve the bioavailability of the cyclic peptide.

### I. Definitions

In order that the present description can be more readily understood, certain terms are first defined. Additional definitions are set forth throughout the detailed description.

Unless otherwise indicated, any atom with unsatisfied valences is assumed to have hydrogen atoms sufficient to satisfy the valences.

The singular forms “a,” “an,” and “the” include plural referents unless the context dictates otherwise. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

The term “or” is a logical disjunction (i.e., and/or) and does not indicate an exclusive disjunction unless expressly indicated such as with the terms “either,” “unless,” “alternatively,” and words of similar effect.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Those of ordinary skill in the art are aware that an amino acid includes a compound represented by the general structure:

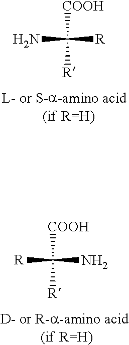

where R and R′ are as discussed herein. Unless otherwise indicated, the term “amino acid” as employed herein, alone or as part of another group, includes, without limitation, an amino group and a carboxyl group linked to the same carbon, referred to as “a” carbon, where R and/or R′ can be a natural or an un-natural side chain, including hydrogen. The absolute “S” configuration at the “a” carbon is commonly referred to as the “L” or “natural” configuration. In the case where both the “R” and the “R′” (prime) substituents equal hydrogen, the amino acid is glycine and is not chiral.

Where not specifically designated, the amino acids described herein can be D- or L-stereochemistry and can be substituted as described elsewhere in the disclosure. It should be understood that when stereochemistry is not specified, the present disclosure encompasses all stereochemical isomeric forms, or mixtures thereof, which produce the desired activity. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

As used herein, the phrase “or a pharmaceutically acceptable salt thereof” refers to at least one compound, or at least one salt of the compound, or a combination thereof. For example, “Compound (I) or a pharmaceutically acceptable salt thereof” includes, but is not limited to, Compound (I), a pharmaceutically acceptable salt of Compound (I), Compound (I) and one or more pharmaceutically acceptable salts of Compound (I), and two or more pharmaceutically acceptable salts of Compound (I).

The term “treating” refers to inhibiting the disease, disorder, or condition, i.e., arresting its development; and (iii) relieving the disease, disorder, or condition, i.e., causing regression of the disease, disorder, and/or condition and/or symptoms associated with the disease, disorder, and/or condition.

### II. Pharmaceutical Compositions

In some aspects, the present disclosure provides compositions comprising cyclic peptides. The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or ignore of the ingredient. Accordingly, the pharmaceutical compositions of the present invention encompass any composition made by mixing a compound of the present invention and a pharmaceutically acceptable carrier. By “pharmaceutically acceptable carrier” it is meant the carrier, diluent or excipient is compatible with the other ingredients of the formulation and not deleterious to the recipient thereof.

In some aspects, the compositions of the disclosure are suitable for oral administration. These compositions can comprise solid, semisolid, gelmatrix or liquid dosage forms suitable for oral administration. As used herein, oral administration includes buccal, lingual, and sublingual administration. Suitable oral dosage forms include, without limitation, tablets, minitablets, bi-layer tablets, capsules, pills, troches, lozenges, pastilles, sachets, pellets, medicated chewing gum, granules, bulk powders, effervescent or non-effervescent powders or granules, solutions, emulsions, suspensions, solutions, wafers, sprinkles, elixirs, syrups or any combination thereof. In some aspects, compositions of the disclosure suitable for oral administration are in the form of a tablet or a capsule. In some aspects, the compound of the disclosure can be formualted tablet. In some aspects, the tablets can be encapsulated into capsules for administration.

The tablets of the disclosure can be in the form of compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which can be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. A film coating can impart the same general characteristics as a sugar coating. Multiple compressed tablets are compressed tablets made by more than one compression cycle, including layered tablets, and press-coated or dry-coated tablets.

In some aspects, the compound of the disclosure can be in the form of a tablet. In some aspects, the compound of the disclosure can be in the form of a compressed tablet. In some aspects, the compound of the disclosure can be in the form of enteric coated tablet.

In some aspects, the compositions of the disclosure can be prepared by dry granulation of the compound of the disclosure with one or more pharmaceutically acceptable carriers, vehicles, and/or excipients. In some aspects, the compositions of the disclosure can be prepared by wet granulation.

In some aspects, the compositions of the disclosure can be in the form of soft or hard capsules, which can be made from gelatin, methylcellulose, starch, and/or calcium alginate. The hard gelatin capsule, also known as the dry-filled capsule (DFC), can comprise two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. In some aspects, tsoft gelatin shells can contain a preservative to prevent the growth of microorganisms. Suitable preservatives include, but are not limited to, those as described herein, including methyl- and propyl-parabens, sorbic acid, and combinations thereof. The liquid, semisolid, and solid dosage forms provided herein can be encapsulated in a capsule. Suitable liquid and semisolid dosage forms include, but are not limited to, solutions and suspensions in propylene carbonate, vegetable oils, triglycerides, and combinations thereof. The capsules can also be coated as known by those of skill in the art in order to modify or sustain dissolution of the active ingredient.

Coloring and flavoring agents can be used in all of the above dosage forms. In addition, flavoring and sweetening agents can be especially useful in the formation of chewable tablets and lozenges.

In certain aspects, the compositions of the disclosure can be formulated as immediate or modified release dosage forms, including delayed-, extended, pulsed-, controlled, targeted-, and programmed-release forms.

The compositions of the disclosure can comprise another active ingredient that does not impair the composition's therapeutic or prophylactic efficacy and/or can comprise a substance that augments or supplements the composition's efficacy.

The compositions described herein are typically part of an admixture with suitable pharmaceutical diluents, excipients, and/or carriers (collectively referred to herein as pharmaceutical carriers) suitably selected with respect to the intended form of administration, and consistent with conventional pharmaceutical practices. For instance, for oral administration in the form of a tablet or capsule, the active drug component can be combined with an oral, non-toxic, pharmaceutically acceptable, inert carriers. Moreover, when desired or necessary, suitable binders, lubricants, surfactants, disintegrating agents, glidants, flavoring agents, and coloring agents can also be incorporated into the mixture. Examples of these types of additives include, but are not limited to, cholesterol, caprylocaproyl macrogol-8 glycerides/caprylocaproyl polyoxyl-8 glycerides, calcium phosphate, calcium sulfate, natural starch, pregelatinized starch, sodium starch glycolate, methylcellulose, microcrystalline cellulose, croscarmellose, croscarmellose sodium, cross-linked sodium carboxymethylcellulose, cross-linked carboxymethylcellulose, cross-linked croscarmellose, cross-linked starch such as sodium starch glycolate, cross-linked polymer such as crospovidone, cross-linked polyvinylpyrrolidone, sodium alginate, clay, gum, silicas, silicon dioxide, talc, starch, magnesium aluminum silicates, sodium lauryl sulfate, sorbitan monooleate, polyoxyethylene sorbitan monooleate, polysorbates, polaxomers, bile salts, glyceryl monostearate, copolymers of ethylene oxide, propylene oxide, stearic acid, calcium hydroxide, talc, corn starch, sodium stearyl fumerate, stearic acid, sodium oleate, sodium stearate, sodium benzoate, sodium acetate, sodium chloride, magnesium stearate, zinc stearate, waxes, talc, and the like, as well as combinations thereof. In some aspects, the compositions described herein comprise cholesterol, caprylocaproyl macrogol-8 glycerides/caprylocaproyl polyoxyl-8 glycerides, silicon dioxide, croscarmellose sodium, and magnesium stearate. In some aspects, the compositions described herein comprise microcrystalline cellulose, mannitol, croscarmelose sodium, silicon dioxide, magnesium stearate, or a combination thereof.

In some aspects, the composition comprises from about 15 wt % to about 80 wt % of one or more fillers. In some aspects, the term “filler” refers to an inactive substance used to make an active ingredient bigger or easier to handle. Examples of fillers include, but are not limited to, lactose, sucrose, microcrystalline cellulose, calcium carbonate, and calcium phosphate. In some aspects, the composition comprises from about 20 wt % to about 75 wt % of one or more fillers. In some aspects, the composition comprises from about 25 wt % to about 70 wt % of one or more fillers. In some aspects, the composition comprises from about 30 wt % to about 65 wt % of one or more fillers. In some aspects, the composition comprises from about 35 wt % to about 60 wt % of one or more fillers. In some aspects, the composition comprises about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, about 40 wt %, about 41 wt %, about 42 wt %, about 43 wt %, about 44 wt %, about 45 wt %, about 46 wt %, about 47 wt %, about 48 wt %, about 49 wt %, about 50 wt %, about 51 wt %, about 52 wt %, about 53 wt %, about 54 wt %, about 55 wt %, about 56 wt %, about 57 wt %, about 58 wt %, about 59 wt %, about 60 wt %, about 61 wt %, about 62 wt %, about 63 wt %, about 64 wt %, about 65 wt %, about 66 wt %, about 67 wt %, about 68 wt %, about 69 wt %, about 70 wt %, about 71 wt %, about 72 wt %, about 73 wt %, about 74 wt %, about 75 wt %, about 76 wt %, about 77 wt %, about 78 wt %, about 79 wt %, or about 80 wt % of one or more fillers.

In some aspects, the one or more fillers comprises microcrystalline cellulose. In some aspects, the composition comprises from about 10 wt % to about 50 wt % microcrystalline cellulose. In some aspects, the composition comprises from about 15 wt % to about 49 wt % microcrystalline cellulose. In some aspects, the composition comprises from about 20 wt % to about 48 wt % microcrystalline cellulose. In some aspects, the composition comprises from about 25 wt % to about 47 wt % microcrystalline cellulose. In some aspects, the composition comprises from about 30 wt % to about 46 wt % microcrystalline cellulose. In some aspects, the composition comprises from about 35 wt % to about 45 wt % microcrystalline cellulose. In some aspects, the composition comprises from about 37 wt % to about 43 wt % microcrystalline cellulose. In some aspects, the composition comprises about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, about 40 wt %, about 41 wt %, about 42 wt %, about 43 wt %, about 44 wt %, about 45 wt %, about 46 wt %, about 47 wt %, about 48 wt %, about 49 wt %, or about 50 wt % microcrystalline cellulose.

In some aspects, the one or more filles comprises mannitol. In some aspects, the composition comprises about 5 wt % to about 30 wt % mannitol. In some aspects, the composition comprises from about 10 wt % to about 27 wt % mannitol. In some aspects, the composition comprises from about 15 wt % to about 25 wt % mannitol. In some aspects, the composition comprises from about 18 wt % to about 22 wt % mannitol. In some aspects, the composition comprises about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, or about 30 wt % mannitol.

In some aspects, the composition comprises about 2 wt % to about 6 wt % of one or more disintegrrants. In some aspects, the term “disintegrant” refers to an agent added to a formulation, in particular a tablet, to promote the break-up of the tablet into smaller fragment in an aqueous environment. Examples of disintegrants include, but are not limited to, croscarmellose sodium, crospovidone, and sodium starch glycolate. In some aspects, the composition comprises about 3 wt % to about 5 wt % of one or more disintegrants. In some aspects, the composition comprises about 2 wt %, about 3 wt %, about 4 wt %, about 5 wt %, or about 6 wt % of one or more disintegrants.

In some aspects, the one or more disintegrants comprises croscarmellose sodium. In some aspects, the composition comprises about 2 wt % to about 6 wt % croscarmellose sodium. In some aspects, the composition comprises about 3 wt % to about 5 wt % croscarmellose sodium. In some aspects, the composition comprises about 2 wt %, about 3 wt %, about 4 wt %, about 5 wt %, or about 6 wt % croscarmellose sodium.

In some aspects, the composition comprises silicon dioxide, talc, calcium silicate, or a mixture thereof. In some aspects, the composition comprises about 1 wt % to about 4 wt % silicon dioxide. In some aspects, the composition comprises about 1 wt % to about 3 wt % silicon dioxide. In some aspects, the composition comprises about 1 wt %, about 2 wt %, about 3 wt %, or about 4 wt % silicon dioxide.

In some aspects, the composition comprises about 1 wt % to about 4 wt % of one or more glidants. In some aspects, the term “glidant” refers to an agent used to increase powder flow. Examples of glidants include, but are not limited to, talc, calckum stearate, sodium stearate, stearic acid, stearyl fumarate, magnesium stearate, and silica. In some aspects, the composition comprises about 1 wt % to about 3 wt % of one or more glidants. In some aspects, the composition comprises about 1 wt %, about 2 wt %, about 3 wt %, or about 4 wt % of one or more glidants.

In some aspects, the composition comprises about 1 wt % to about 4 wt % magnesium stearate. In some aspects, the composition comprises about 1 wt % to about 3 wt % magnesium stearate. In some aspects, the composition comprises about 1 wt %, about 2 wt %, about 3 wt %, or about 4 wt % magnesium stearate.

In still other aspects, using standard coating procedures, such as those described in Remington's Pharmaceutical Sciences, 23^{rd} Edition (2020), a film coating can be provided around the formulation of the compounds described herein.

Dosage forms (pharmaceutical compositions) suitable for administration can contain from about 1 milligram to about 300 milligrams of active ingredient per dosage unit. In these pharmaceutical compositions the active ingredient will ordinarily be present in an amount of about 0.5-95% by weight based on the total weight of the composition. In some aspects, dosage forms suitable for administration can contain from about 10 to about 240 milligrams of active ingredient per dosage unit. In some aspects, dosage forms suitable for administration can contain about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, or about 240 mg of active ingredient per dosage unit.

In some aspects, the present disclosure provides pharmaceutical compositions which comprise a cyclic peptide, or a pharmaceutically acceptable salt thereof, as described herein, a salt of decanoic acid, and at least one pharmaceutical acceptable carrier. In some aspects, the present disclosure provides pharmaceutical compositions which comprise a cyclic peptide, or a pharmaceutically acceptable salt thereof, as described herein, sodium decanoate, and at least one pharmaceutical acceptable carrier.

The present disclosure provides a pharmaceutical composition comprising a cyclic peptide and a salt of decanoic acid. In some aspects the salt is sodium decanoate, potassium decanoate, lithium decanoate, or magnesium decanoate. In some aspects the salt is sodium decanoate. In certain aspects, the cyclic peptide backbone can comprise 2 to 20 amino acids. In certain aspects, the cyclic peptide backbone can comprise 4 to 18 amino acids. In certain aspects, the cyclic peptide backbone can comprise 6 to 16 amino acids. In certain aspects, the cyclic peptide backbone can comprise 8 to 14 amino acids. In certain aspects, the cyclic peptide backbone can comprise 10 to 14 amino acids. In certain aspects, the cyclic peptide backbone can comprise 2, 3, 4, 5, 6, 7, 8, 9, 10, 11, 12, 13, 14, 15, 16, 17, 18, 19, or 20 amino acids.

In certain aspects, the cyclic peptide can comprise Compound (I) having the formula:

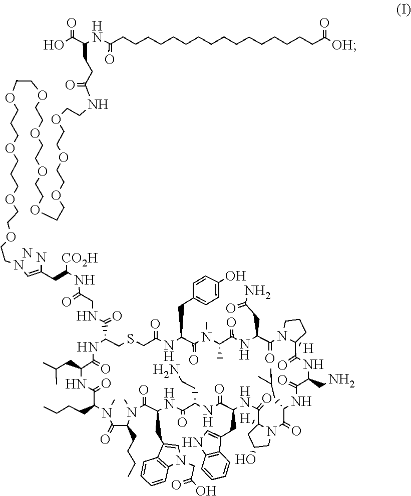 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

The pharmaceutical compounds of the disclosure can include one or more pharmaceutically acceptable salts. A “pharmaceutically acceptable salt” refers to a salt that retains the desired biological activity of the parent compound and does not impart any undesired toxicological effects (see e.g. Remington's Pharmaceutical Sciences, 23^{rd} Edition (2020)). The salts can be obtained during the final isolation and purification of the compounds described herein, or separately by reacting a free base function of the compound with a suitable acid or by reacting an acidic group of the compound with a suitable base. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

In certain aspects, the cyclic peptide comprises about 0.1 wt % to about 25 wt % of the pharmaceutical composition. In some aspects, the cyclic peptide comprises about 0.1 wt % to about 20 wt % of the pharmaceutical composition. In some aspects, the cyclic peptide comprises about 0.5 wt % to about 10 wt % of the pharmaceutical composition. In some aspects, the cyclic peptide comprises about 0.8 wt % to about 5 wt % of the pharmaceutical composition. In some aspects, the cyclic peptide comprises about 6.0 wt % to about 9.5 wt % of the pharmaceutical composition. In some aspects, the cyclic peptide comprises about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.5 wt %, about 1.6 wt %, about 1.7 wt %, about 1.8 wt %, about 1.9 wt %, about 2 wt %, about 2.5 wt %, about 3 wt %, about 3.0 wt %, about 3.2 wt %, about 3.3 wt %, about 3.4 wt %, about 3.5 wt %, about 4.0 wt %, about 4.5 wt %, about 5.0 wt %, about 5.5 wt %, about 6.0 wt %, about 6.5 wt %, 6.6 wt %, 6.7 wt %, 6.8 wt %, 6.9 wt %, about 7.0 wt %, about 7.5 wt %, about 8.0 wt %, about 8.1 wt %, about 8.2 wt %, about 8.3 wt %, about 8.4 wt %, about 8.5%, about 9.0 wt %, about 9.5 wt %, about 10.0 wt %, about 11.0 wt %, about 12.0 wt %, about 13.0 wt %, about 14.0 wt %, about 15.0 wt %, about 16.0 wt %, about 17.0 wt %, about 18.0 wt %, about 19.0 wt %, or about 20.0 wt % of the composition.

The pharmaceutical compositions described herein comprise sodium decanoate. In certain aspects, the sodium decanoate comprises about 10 wt % to about 80 wt % of the composition. In certain aspects, the sodium decanoate comprises about 15 wt % to about 70 wt % of the composition. In certain aspects, the sodium decanoate comprises about 20 wt % to about 60 wt % of the composition. In certain aspects, the sodium decanoate comprises about 20 wt % to about 55 wt % of the composition. In certain aspects, the sodium decanoate comprises about 20 wt % to about 30 wt % of the composition. In certain aspects, the sodium decanoate comprises about 27 wt % to about 28 wt % of the composition. In certain aspects, the sodium decanoate comprises about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, about 40 wt %, about 41 wt %, about 42 wt %, about 43 wt %, about 44 wt %, about 45 wt %, about 46 wt %, about 47 wt %, about 48 wt %, about 49 wt %, about 50 wt %, about 51 wt %, about 52 wt %, about 53 wt %, about 54 wt %, about 55 wt %, about 56 wt %, about 57 wt %, about 58 wt %, about 59 wt %, about 60 wt %, about 61 wt %, about 62 wt %, about 63 wt %, about 64 wt %, about 65 wt %, about 66 wt %, about 67 wt %, about 68 wt %, about 69 wt %, about 70 wt %, about 71 wt %, about 72 wt %, about 73 wt %, about 74 wt %, about 75 wt %, about 76 wt %, about 77 wt %, about 78 wt %, about 79 wt %, or about 80 wt % of the composition.

In some aspects, the sodium decanoate is amorphous, produced by spray drying or another suitable process. In some aspects, the sodium decanoate is crystallized. Without being bound by a particular theory, the crystallized sodium decanoate can in some cases provide improved physical properties, such as favorable flow or less sticking than when the sodium decanoate is spray dried.

In certain aspects, the present disclosure provides a pharmaceutical composition comprising about 0.1 wt % to about 25 wt % of a cyclic peptide, about 10 wt % to about 80 wt % of sodium decanoate, about 10 wt % to about 50 wt % microcrystalline cellulose, about 5 wt % to about 30 wt % mannitol, about 2 wt % to about 6 wt % croscarmellose sodium, about 1 wt % to about 4 wt % silicon dioxide, and about 1 wt % to about 4 wt % magnesium stearate. In some aspects, the cyclic peptide is Compound (I).

In certain aspects, the present disclosure provides a pharmaceutical composition comprising about 0.1 wt % to about 20 wt % of a cyclic peptide, about 15 wt % to about 70 wt % of sodium decanoate, about 15 wt % to about 49 wt % microcrystalline cellulose, about 10 wt % to about 27 wt % mannitol, about 3 wt % to about 5 wt % croscarmellose sodium, about 1 wt % to about 3 wt % silicon dioxide, and about 1 wt % to about 3 wt % magnesium stearate. In some aspects, the cyclic peptide is Compound (I).

In certain aspects, the present disclosure provides a pharmaceutical composition comprising about 0.8 wt % of a cyclic peptide, about 27.5 wt % of sodium decanoate, about 42 wt % microcrystalline cellulose, about 21 wt % mannitol, about 4 wt % croscarmellose sodium, about 2 wt % silicon dioxide, and about 2 wt % magnesium stearate. In some aspects, the cyclic peptide is Compound (I).

In certain aspects, the present disclosure provides a pharmaceutical composition comprising about 1.7 wt % of a cyclic peptide, about 27.5 wt % of sodium decanoate, about 42 wt % microcrystalline cellulose, about 21 wt % mannitol, about 4 wt % croscarmellose sodium, about 2 wt % silicon dioxide, and about 2 wt % magnesium stearate. In some aspects, the cyclic peptide is Compound (I).

In certain aspects, the present disclosure provides a pharmaceutical composition comprising about 3.3 wt % of a cyclic peptide, about 27.5 wt % of sodium decanoate, about 41 wt % microcrystalline cellulose, about 20 wt % mannitol, about 4 wt % croscarmellose sodium, about 2 wt % silicon dioxide, and about 2 wt % magnesium stearate. In some aspects, the cyclic peptide is Compound (I).

In certain aspects, the present disclosure provides a pharmaceutical composition comprising about 6.7 wt % of a cyclic peptide, about 27.5 wt % of sodium decanoate, about 38.5 wt % microcrystalline cellulose, about 19 wt % mannitol, about 4 wt % croscarmellose sodium, about 2 wt % silicon dioxide, and about 2 wt % magnesium stearate. In some aspects, the cyclic peptide is Compound (I).

In certain aspects, the present disclosure provides a pharmaceutical composition comprising about 8.3 wt % of a cyclic peptide, about 27.5 wt % of sodium decanoate, about 37 wt % microcrystalline cellulose, about 19 wt % mannitol, about 4 wt % croscarmellose sodium, about 2 wt % silicon dioxide, and about 2 wt % magnesium stearate. In some aspects, the cyclic peptide is Compound (I).

Also within the scope of the present disclosure are kits comprising the compositions of the disclosure and instructions for use. The kit can further contain additional reagents. Kits typically include a label indicating the intended use of the contents of the kit and instructions for use. The term label includes any writing, or recorded material supplied on or with the kit, or which otherwise accompanies the kit.

### III. Methods of Using

The present disclosure provides pharmaceutical formulations that improve the bioavailibity of cyclic peptides. In certain aspects, the bioavailability is improved at least by about 0.1%, at least by about 0.2%, at least by about 0.3%, at least by about 0.4%, at least by about 0.5%, at least by about 0.6%, at least by about 0.7%, at least by about 0.8%, at least by about 0.9%, or at least about 1.0%.

Administration of a cyclic peptide described herein includes, without limitation, administration of a therapeutically effective amount of the compound. The term “therapeutically effective amount” as used herein refers, without limitation, to an amount of a cyclic peptide to treat a condition treatable by administration of a composition comprising the compound. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration. Thus, it is not useful to specify an exact effective amount in advance.

In another aspect, the disclosure pertains to methods of inhibiting growth of tumor cells in a subject using the pharmaceutical compositions of the present disclosure. In some aspects, the cyclic peptide is capable of binding to PD-L1, disrupting the interaction between PD-L1 and PD-1, competing with the binding of PD-L1 with anti-PD-1 monoclonal antibodies that are known to block the interaction with PD-1, enhancing CMV-specific T cell IFNγ secretion, and enhancing HIV-specific T cell IFNγ secretion. As a result, in some aspects, the cyclic peptides of the present disclosure are useful for modifying an immune response, treating diseases such as cancer, infectious disease, and/or septic shock, stimulating a protective autoimmune response or to stimulate antigen-specific immune responses.

Cancers whose growth can be inhibited using the pharmaceutical compositions of the present disclosure include, but are not limited to, cancers typically responsive to immunotherapy. Representative examples include melanoma (e.g., metastatic malignant melanoma), renal cell carcinoma, prostate cancer (including, but not limited to, castration-resistent prostate cancer), breast cancer, colon cancer and lung cancer (including, but not limited to, squamous non-small cell lung cancer, non-squamous non-small cell lung cancer). Examples of other cancers that can be treated using the methods of the present disclosure include bone cancer, hepatocellular carcinoma, pancreatic carcinoma, skin cancer, squamous cell carcinoma of the head and neck, cutaneous or intraocular malignant melanoma, uterine cancer, ovarian cancer, rectal cancer, cancer of the anal region, gastric cancer, gastrointestinal tract cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, carcinomas of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, hematological malignancies (such as chronic or acute leukemias including acute myeloid leukemia, chronic myeloid leukemia, acute lymphoblastic leukemia, chronic lymphocytic leukemia), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The pharmaceutical compositions described herein can also be useful for treatment of metastatic cancers.

The pharmaceutical compositions described herein can enhance, stimulate, and/or increase an immune response in a subject in need thereof. As used herein, the term “immune response” refers to the action of, for example, lymphocytes, antigen presenting cells, phagocytic cells, granulocytes, and soluble macromolecules produced by the above cells or the liver (including macrocyclic peptides, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues. In certain aspects, the immune response can be generated by the innanate immune system. In certain aspects, the immune response can be generated by the adaptive immune system. In certain embodiments, the immune response can be generated by the innate immune system and the adaptive immune system.

The pharmaceutical compositions described herein can be delivered by numerous methods, after some modifications, including, but not limited to, orally, subcutaneously, intramuscularly, intraduodenally, or intravenously. The composition can be formulated according to the route of administration based on acceptable pharmacy practice (Fingl et al., in The Pharmacological Basis of Therapeutics, Chapter 1, p. 1 (1975); Remington's Pharmaceutical Sciences, 18th Edition, Mack Publishing Co., Easton, PA (1990)).

The dosage regimen for the compositions described herein will, of course, vary depending upon known factors, such as the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the disease state.

By way of general guidance, the daily oral dosage of the active ingredient, when used for the indicated effects, will range between about 0.001 to 500 mg/kg of body weight, preferably between about 0.01 to 100 mg/kg of body weight per day, and most preferably between about 0.1 to 20 mg/kg/day. Intravenously, the daily dosage of the active ingredient when used for the indicated effects will range between 0.001 ng to 100.0 ng per min/per Kg of body weight during a constant rate infusion. Such constant intravenous infusion can be preferably administered at a rate of 0.01 ng to 50 ng per min per Kg body weight and most preferably at 0.01 ng to 10.0 mg per min per Kg body weight. The compositions described herein may be administered in a single daily dose, or the total daily dosage may be administered in divided doses of two, three, or four times daily.

### Biological Activity

Compound (I) (prepared according to the procedure described in U.S. Pat. No. 9,856,292).

### Example 1

### Preparation of Compound (I) Enteric Coated Tablets of 1 mg Strength for 12 mg Dose (12 Tablets Total)

The composition of uncoated core tablets is shown in Table 1. Cholesterol was weighed and dissolved in the Labrasol ALF in a glass vial. Sodium decanoate was combined with the silicon dioxide in a mortar and blended using a pestle. The solution containing the dissolved cholesterol in Labrasol ALF was added to the blend of sodium decanoate and silicon dioxide. The vial containing the solution was rinsed with 1 g of ethyl alcohol, and the rinsate was added to the blend. In another vial, Compound (I) was dissolved in 8 g of ethyl alcohol and added to the blend. The vial containing the solution was rinsed with 1 g of ethyl alcohol, and the rinsate was added to the blend. The blend containing all formulation components was mixed with a pestle in the mortar until the blend was visually homogenous. The mixture was placed in an aluminum tray and dried in a vacuum oven at 50° C. overnight. The dried granules were then placed in a mortar and gently ground and passed through a 30-mesh screen. The screened granules were then added to a glass bottle along with the croscarmellose sodium, and the mixture was blended for 10 minutes using a Turbula mixer (32 RPM, 10 minutes). Magnesium stearate was then added to the glass bottle and the final mixture was blended for 5 minutes using a Turbula mixer (32 RPM, 5 minutes). The powder blend was compressed using a 7/32″ (5.55 mm) round standard concave tooling at 400 lb compression force to form tablets at a target weight of 70 mg.

TABLE 1
Composition of Uncoated Core Tablets

| | | |
|---|---|---|
| Ingredient | % (w/w) | Dose Amt. (mg) |

Intra-granular Components

| | | |
|---|---|---|
| Compound (I)* | 1.51 | 12 |
| Sodium Decanoate | 49.74 | 396 |
| Silicon Dioxide** | 22.61 | 180 |
| Labrasol ALF | 25.63 | 204 |
| Cholesterol | 0.51 | 4.08 |
| Ethyl alcohol*** | (50.00) | N/A |

Extra-granular Components

| | | |
|---|---|---|
| Croscarmellose Sodium | 5.00 | 37.56 |
| Magnesium Stearate | 0.75 | 5.628 |
| Total | 100.00 | 796.08 |

*Amount of Compound (I) after potency correction with an assay “as is” value of 89%
**Amount of silicon dioxide is adjusted to compensate for potency correction.
***Ethyl alcohol is removed during a drying step.

The composition of the seal coated tablet is shown in Table 2. An aqueous suspension of Opadry 03K was prepared at 7.5% solid content. The uncoated core tablets and placebo tablets were placed in a Vector 0.5-L coating pan. The tablets were pan coated to achieve 2% target weight gain.

TABLE 2
Composition of Seal Coated Tablets

| Ingredient | % (w/w) |
|---|---|
| Uncoated core tablets and placebo tablets* | 98.04 |
| Opadry 03K19229 | 1.96 |
| Total | 100.00 |

*Placebo tablets are added to achieve a suitable batch size for coating process.

The composition of the enteric coated tablet is shown in Table 3. An aqueous suspension of Acryl EZE II was prepared at 10% solid content. Seal coated tablets and placebo tablets were added to a Vector 0.5-L coating pan. The tablets were pan coated to achieve 7% target weight gain. Six enteric coated tablets were encapsulated in a size #00 hard gelatin capsule for the cyno PK study. Each cyno monkey was dosed with 2 capsules (12 enteric coated tablets).

TABLE 3
Composition of Enteric Coating

| Ingredient | % (w/w) |
|---|---|
| Seal coated tablets and placebo tablets* | 93.46 |
| Acryl-EZE II 493Z180022 White | 6.54 |
| Total | 100.00 |

*Placebo tablets are added to achieve a suitable batch size for coating process.

### Example 2

### Determination of Oral Absorption of Prepared Formulations of the Compound (I) in Cynomolgus Monkeys

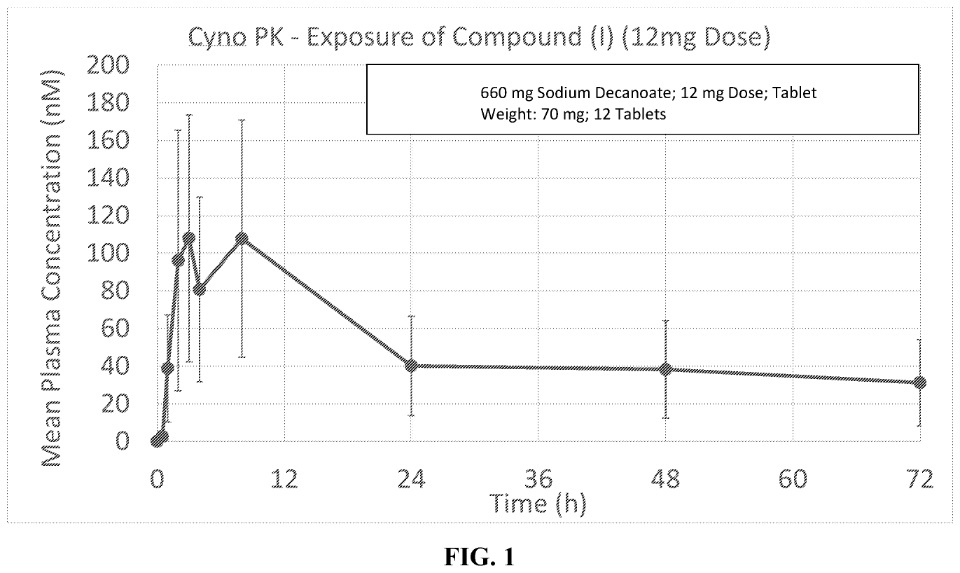

Fasted male cyno monkeys (n=3) were dosed by oral route followed by gavage flush with water to facilitate gastro-intestinal dissolution. Blood samples were taken at predetermined times over 72 hours post-dose to measure comparative pharmacokinetics between the formulations. Due to the long half-life these were conducted in a parallel group design in different sets of cyno monkeys. The mean exposure is shown in FIG. 1 with error bars showing the standard deviation. As shown in both the figure and Table 4, the formulation provided good plasma concentration of Compound (I) over an extended period of time.

TABLE 4
Pharmacokinetic Profile of Formulated Tablets

| | | | | | | | | | | | | BA (%) | | |
|---|---|---|---|---|---|---|---|---|---|---|---|---|---|---|
| | | | | | | | | | | | | using | | |
| | | Cmax (nM) | | | AUC_{0-24 h} (nM · h) | | BA (%) | | | AUC_{inf} (nM · h) | | AUC_{Inf} | | |
| | Dose | | Std | Tmax (h) | | Std | | Std | CV | | Std | | Std | CV |
| | (mg) | Mean | Dev | (Median) | Mean | Dev | Mean | Dev | (%) | Mean | Dev | Mean | Dev | (%) |
| 0.1 mg/kg IV | 0.5 | 798.33 | 78.80 | 0.3 | 47613.51 | 2131.26 | 100.00 | n/a | 4.48 | 51965.95 | 2466.56 | 100.00 | n/a | 4.7 |
| Example 1 | 12 | 108.73 | 64.40 | 3.0 | 1834.76 | 1120.09 | 0.74 | 0.45 | 61.05 | 10077.62 | 8512.43 | 0.81 | 0.68 | 84.5 |

### Example 3

### Preparation of Compound (I) Enteric Coated Tablets of 2 mg Strength for 20 mg Dose (10 Tablets Total) with 660 mg of Sodium Decanoate

The composition of uncoated core tablets is shown in Table 5. Compound (I) and sodium decanoate were combined using a mortar and a pestle. The powder mixture was transferred into a glass bottle and blended using a Turbular mixture (32 RPM, 5 minutes). Magnesium stearate was added into the same bottle and gently mixed using a spatula. The powder mixture was blended using a Turbular mixture (32 RPM, 5 minutes). The powder blend was compressed using a 7/32-inch (5.55 mm) round standard concave tooling at 300 lb compression force to form tablets at a target weight of 70 mg.

TABLE 5
Composition of Uncoated Core Tablets

| Ingredient | % (w/w) | Dose Amt. (mg) |
|---|---|---|
| Compound (I)* | 2.89 | 20** |
| Sodium decanoate | 95.51 | 660 |
| Magnesium stearate | 1.59 | 11 |
| Total** | 100 | 691 |

*Amount of Compound (I) is adjusted based on Assay “as is” of 89%.
**Total dose amount is adjusted after potency correction.

The composition of seal coated tablet is shown in Table 6. An aqueous suspension of Opadry 03K was prepared at 7.5% solid content. The uncoated core tablets and placebo tablets were placed in a Vector 0.5-L coating pan. The tablets were pan coated to achieve 2% target weight gain.

TABLE 6
Composition of Seal Coated Tablets

| Ingredient | % (w/w) |
|---|---|
| Uncoated core tablets and placebo tablets* | 98.04 |
| Opadry 03K19229 Clear | 1.96 |
| Total | 100.00 |

*Placebo tablets are added to achieve a suitable batch size for coating process.

The composition of enteric coated tablet is shown in Table 7. An aqueous suspension of Acryl-EZE II was prepared at 10% solid content. Compound (I) seal coated tablets and placebo tablets were added to a Vector 0.5-L coating pan. Tablets were pan coated to achieve 7% target weight gain. Five enteric coated tablets were encapsulated into a size #0 hard gelatin capsule for the dog PK study. Each dog was dosed with 2 capsules (10 enteric coated tablets).

TABLE 7
Composition of Enteric Coated Tablets

| Ingredient | % (w/w) |
|---|---|
| Seal coated tablets and placebo tablets* | 93.58 |
| Acryl-EZE II 493Z180022 White | 6.52 |
| Total | 100.00 |

*Placebo tablets are added to achieve a suitable batch size for coating process.

### Example 4

### Dog pK Study

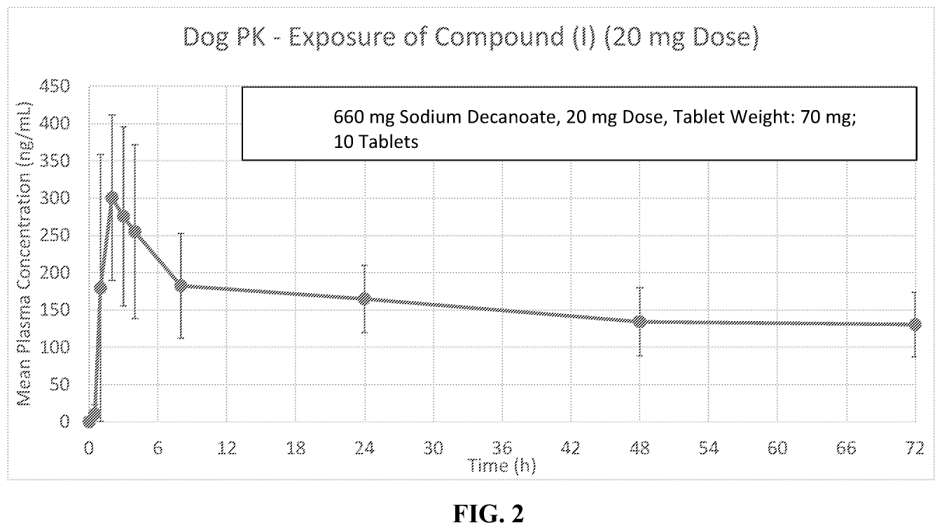

Fasted dogs (4), pre-treated with pentagastrin, were dosed with a single 20 mg dose (2×10 mg capsules) of Compound (I). Blood samples were withdrawn over a period of 72 hours. FIG. 2 shows the mean exposure. Error bars describe standard deviation. As shown in the figure and also in Table 8, dogs had a consistent plasma concentration of Compound (I) over the 72-hour period.

TABLE 8
Pharmacokinetic Profile of Formulated Tablets

| | | Cmax | | | AUC_{0-72 h} | | | | | AUC_{inf} | | | | |
|---|---|---|---|---|---|---|---|---|---|---|---|---|---|---|
| | | (ng/mL) | | Tmax | (ng · h/mL) | | BA_{0-72\h} (%) | | | (ng · h/mL) | | BA_{inf} (%) | | |
| | Dose | | Std | (h) | | Std | | Std | CV | | Std | | Std | CV |
| | (mg) | Mean | Dev | (Median) | Mean | Dev | Mean | Dev | (%) | Mean | Dev | Mean | Dev | (%) |
| 10 mg IV | 10 | 25466.29 | 1903.34 | 0.2 | 832979.81 | 115022.86 | 100.00 | n/a | 13.81 | 1198538.03 | 115842.07 | 100.00 | n/a | 9.67 |
| Control | | | | | | | | | | | | | | |
| Example 3 | 20 | 307.42 | 116.14 | 2.0 | 11264.78 | 2862.54 | 0.68 | 0.17 | 25.41 | 37371.35 | 23469.22 | 1.56 | 0.98 | 62.80 |

### Example 5

### Effect of Sodium Decanoate Amount and Number of Tablets on Performance of Compound (I) Formulation in Dogs

This study was initiated to determine if oral bioavailability can be impacted by (1) changing the amount of sodium decanoate from 330 mg to 660 mg at 100 mg dose; and (2) the number of tablets administered to achieve a selected dose of 100 mg.

### Example 5A

### Preparation of Compound (I) Enteric Coated Tablets of 2.083 mg Strength for 100 mg Dose (48 Tablets Total) with 330 mg of Sodium Decanoate

The composition of uncoated core tablets is shown in Table 9. Compound (I), sodium decanoate, microcrystalline cellulose, mannitol, croscarmellose sodium, and silicon dioxide, were added to a glass bottle and blended using a Turbular mixer at 25 RPM for 10 minutes. The blended powder was passed through a 20-mesh screen and blended again at 25 RPM for 10 minutes. Magnesium stearate was added to the mixture and blended at 25 RPM for an additional 3 minutes to form pre-blend.

TABLE 9
Formulation Composition of Uncoated Core Tablets

| | | |
|---|---|---|
| Ingredient | % (w/w) | Dose Amt. (mg) |

Intra-granular Components

| | | |
|---|---|---|
| Compound (I)* | 8.33 | 100 |
| Sodium decanoate | 27.5 | 330 |
| Microcrystalline cellulose** | 37.45 | 449.33 |
| Mannitol | 18.72 | 224.67 |
| Croscarmellose sodium | 2 | 24 |
| Silicon dioxide | 2 | 24 |
| Magnesium stearate | 1 | 12 |

Extra-granular Components

| | | |
|---|---|---|
| Croscarmellose sodium | 2 | 24 |
| Magnesium stearate | 1 | 12 |
| Total | 100 | 1,200 |

*Amount of Compound (I) is adjusted based on with Assay “as is” of 88.6%
**Microcrystalline cellulose is adjusted to compensate for potency correction.

The pre-blend was compressed to form compacts at a target weight of 1,000 mg and a target solid fraction of 0.6-0.7 using ¾-inch (19.05 mm) round flat faced tooling. The compacts were added to a mortar and pestle setup and gently ground with 10 compacts at a time. The ground material was passed through a 10-mesh screen and the remaining material was pressed through the screen. The resulting granules were passed through an 18-mesh screen and the remaining material was pressed through the screen. The screened granules and extra-granular croscarmellose sodium were added to a glass bottle and blended at 25 RPM for 10 minutes. Extra-granular magnesium stearate was added and blended at 25 RPM for 3 minutes to form the final blend.

The final blend from was compressed to form tablets at a target weight of 25 mg and a target tensile strength of 1.5-2 MPa using ⅛-inch (3.175 mm) round standard concave multi-tip (7 tips) tooling.

The tablets were coated in a 0.5-L pan LDCS Hi-Coater with 2 layers of coating. The first layer was seal coating with Opadry 03K aqueous suspension (10% w/w solid content) to achieve 2% target weight gain. The second layer was enteric coating with Acryl-EZE II aqueous suspension (20% w/w solid content) to achieve 10% target weight gain. Twenty-four enteric coated tablets were encapsulated into a size #00 hard gelatin capsule for the dog PK study. Each dog was dosed with 2 capsules (48 enteric coated tablets).

### Example 5B

### Preparation of Compound (I) Enteric Coated Tablets of 2.083 mg Strength for 100 mg Dose (48 Tablets Total) with 660 mg of Sodium Decanoate

TABLE 10
Formulation Composition of Uncoated Core Tablets

| | | |
|---|---|---|
| Ingredient | % (w/w) | Dose Amt. (mg) |

Intra-granular Components

| Compound (I) | 9.41* | 100 | (112.87*) |
|---|---|---|---|
| Sodium Decanoate | 55 | 660 | |
| Microcrystalline Cellulose | 16.06 | 199.2 | (192.78**) |
| Mannitol | 8.03 | 102.78 | (96.36**) |

| | | |
|---|---|---|
| Croscarmellose Sodium | 2.5 | 30 |
| Hydroxypropyl Cellulose | 3.5 | 42 |
| Water for Granulation | (16.0)*** | N/A |

Extra-granular Components

| | | |
|---|---|---|
| Croscarmellose Sodium | 2.5 | 30 |
| Silicon Dioxide | 2.0 | 24 |
| Magnesium Stearate | 1.0 | 12 |
| Total | 100 | 1,200 |

*Amount of Compound (I) after potency correction with Assay “as is” of 88.6%
**Microcrystalline cellulose and mannitol are adjusted to compensate for potency correction.
***Based on total batch size. Water is removed during a drying step

The composition of uncoated core tablets is shown in Table 10. All intra-granular ingredients were added to a 250 mL MiPro granulator bowl and blended for 3 minutes at an impeller speed of 1300 RPM and a chopper speed of 300 RPM. Water was added at a rate of 0.5 mL/minute, while the impeller and chopper speeds were maintained. After the completion of water addition, the granulated blend was mixed for 3 additional minutes at the same impeller and chopper speeds. The resultant granules were then passed through an 8-mesh screen and placed in an aluminum pan. The pan was then placed into a convection oven at 40° C. for 1 hour. The dried granules were then passed through a comill equipped with a 45R screen.

The milled granules and extra-granular croscarmellose sodium were added to a glass bottle and blended at 25 RPM for 10 minutes. Extra-granular magnesium stearate was added and blended at 25 RPM for 3 minutes to form final blend. The final blend was compressed to form tablets at a target weight of 25 mg and target tensile strength of 1.5-2 MPa using ⅛″ (3.175 mm) multi-tip (7 tips) round standard concave tooling.

The tablets were coated in a 0.5-L pan LDCS Hi-Coater with 2 layers of coating. The first layer was seal coating with an Opadry 03K aqueous suspension (10% w/w solid content) to achieve 2% target weight gain. The second layer was an enteric coating with Acryl-EZE II aqueous suspension (20% w/w solid content) to achieve 10% target weight gain. Twenty-four enteric coated tablets were encapsulated into a size #00 hard gelatin capsule for the dog PK study. Each dog was dosed with 2 capsules (48 enteric coated tablets).

### Example 5C

### Preparation of Compound (I) Enteric Coated Tablets of 10 mg Strength for 100 mg Dose (10 Tablets Total) with 660 mg of Sodium Decanoate

The composition of uncoated core tablets is shown in Table 11. Compound (I), sodium decanoate, microcrystalline cellulose, mannitol, croscarmellose sodium, and silicon dioxide, were added to a glass bottle and blended using a Turbular mixer at 25 RPM for 10 minutes. The blended material was passed through a 20-mesh screen and blended again at 25 RPM for 10 minutes. Magnesium stearate was added to the mixture and blended at 25 RPM for an additional 3 minutes to form pre-blend.

TABLE 11
Formulation Composition of Uncoated Core Tablets

| | | |
|---|---|---|
| Ingredient | % (w/w) | Dose Amt. (mg) |

Intra-granular Components

| | | |
|---|---|---|
| Compound (I)* | 8.33 | 100 |
| Sodium decanoate | 55 | 660 |
| Microcrystalline cellulose** | 14.67 | 176 |
| Mannitol | 14 | 168 |
| Croscarmellose sodium | 2 | 24 |
| Silicon dioxide | 2 | 24 |
| Magnesium stearate | 1 | 12 |

Extra-granular Components

| | | |
|---|---|---|
| Croscarmellose sodium | 2 | 24 |
| Magnesium stearate | 1 | 12 |
| Total | 100 | 1,200 |

*Amount of Compound (I) is adjusted based on with Assay “as is” of 86.6%
**Microcrystalline cellulose is adjusted to compensate for potency correction.

The pre-blend was compressed to form compacts at a target weight of 400 mg and a target solid fraction of 0.6-0.7 using 11.28 mm round flat faced tooling. The compacts were milled using an oscillator equipped with 4 mm and 1 mm screens. The screened granules and extra-granular croscarmellose sodium were added to a glass bottle and blended at 25 RPM for 10 minutes. Extra-granular magnesium stearate was added and blended at 25 RPM for 3 minutes to form the final blend. The final blend was compressed to form tablets at a target weight of 120 mg and a target tensile strength of 1.5-2 MPa using 7/32″ (5.55 mm) round standard concave tooling.

The tablets were coated in a 0.5-L pan LDCS Hi-Coater with 2 layers of coating. The first layer was seal coating with Opadry 03K aqueous suspension (10% w/w solid content) to achieve 2% target weight gain. The second layer was enteric coating with Acryl-EZE II aqueous suspension (20% w/w solid content) to achieve 7% target weight gain. Five enteric coated tablets were encapsulated into a size #00 hard gelatin capsule for the dog PK study. Each dog was dosed with 2 capsules (10 enteric coated tablets).

### Example 5D

### Preparation of Compound (I) Enteric Coated Tablets of 50 mg Strength for 100 mg Dose (2 Tablets Total) with 660 mg of Sodium Decanoate

The final blend from example 5C was compressed to form tablets at a target weight of 600 mg and a target tensile strength of 1.5-2 MPa using 15.3 mm×8 mm oval standard concave tooling. The tablets were coated in a 0.5-L pan LDCS Hi-Coater with 2 layers of coating. The first layer was seal coating with Opadry 03K aqueous suspension (10% w/w solid content) to achieve 2% target weight gain. The second layer was enteric coating with Acryl-EZE II aqueous suspension (20% w/w solid content) to achieve 7% target weight gain. Each dog was dosed with 2 enteric coated tablets.

### Example 5E

### Dog pK Study

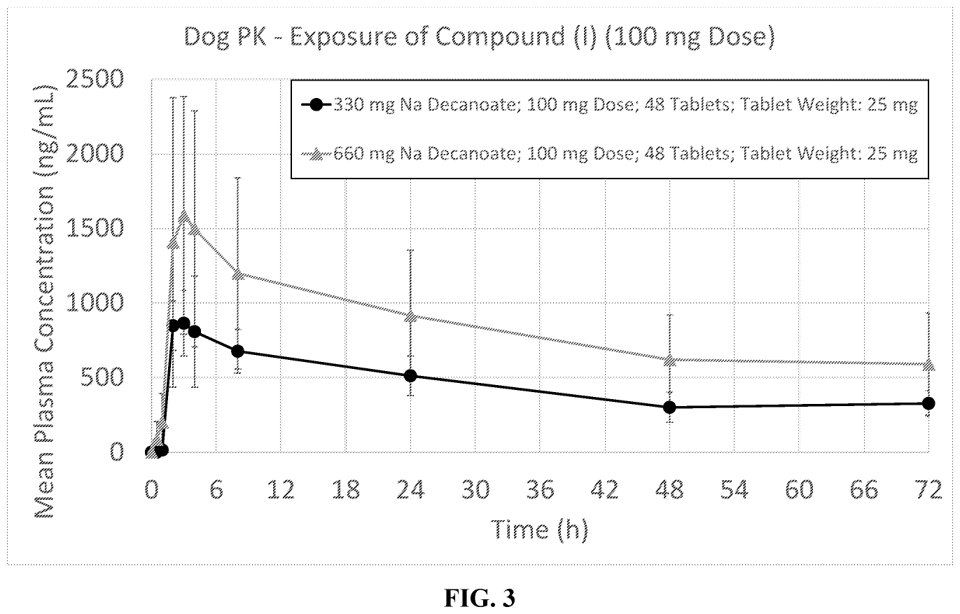

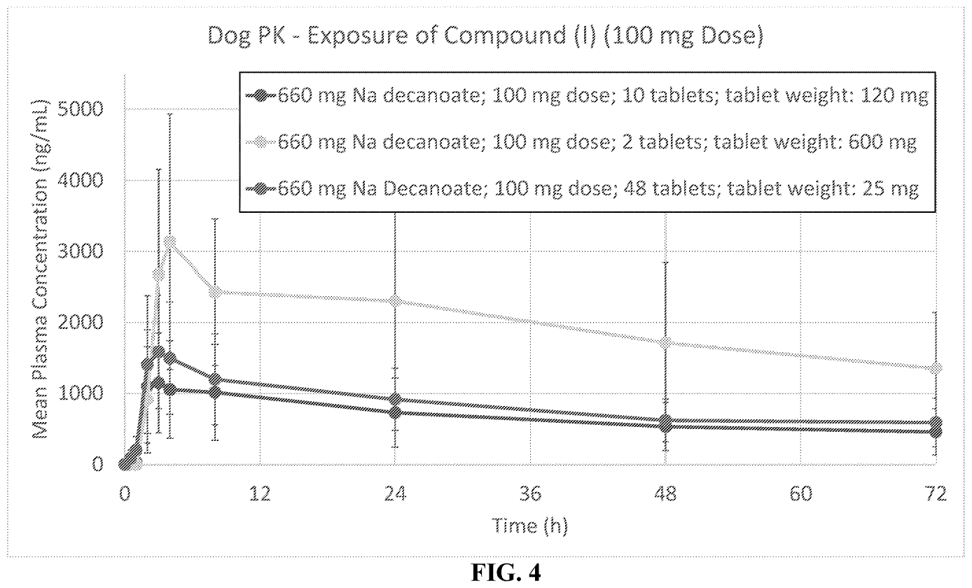

Fasted dogs (4), pre-treated with pentagastrin, were dosed with different formulations of a single 100 mg dose of the Compound (I). Blood samples were withdrawn over a period of 72 hours. FIGS. 3 and 4 show the mean exposure. Error bars describe standard deviation. As shown in the figures and in Table 12, dogs had a consistent plasma concentration of Compound (I) over the 72-hour period.

As shown in FIG. 3, high decanoate levels (660 mg) were needed for enhanced permeation of the API. Decreasing the amount of sodium decanoate decreased PK exposure at 330 mg of sodium decanoate. As shown in FIG. 4, for the 660 mg sodium decanoate formulation, decreasing the number of tablets from 48 or 10 to 2 resulted in an improved PK exposure. Table 12 summarizes all of the PK data for all the formulations tested in Example 5.

TABLE 12
Pharmacokinetic Profile of Formulated Tablets

| | | # of | | | | | | | |
|---|---|---|---|---|---|---|---|---|---|
| | | Tablets | Cmax | | | AUC_{0-72 h} | | | |
| | | for a | (ng/mL) | | Tmax | (ng · h/mL) | | BA_{0-72\h} (%) | |
| | Dose | Given | | Std | (h) | | Std | | Std |
| | (mg) | Dose | Mean | Dev | (Median) | Mean | Dev | Mean | Dev |
| 10 mg IV | 10 | N/A | 25466.29 | 1903.34 | 0.2 | 832979.81 | 115022.86 | 100.00 | n/a |
| Control | | | | | | | | | |
| Example | 100 | 48 | 979.53 | 248.83 | 3.0 | 31905.92 | 8366.31 | 0.38 | 0.10 |
| 5A | | | | | | | | | |
| Example | 100 | 48 | 1667.07 | 845.17 | 2.5 | 59227.05 | 29804.20 | 0.71 | 0.36 |
| 5B | | | | | | | | | |
| Example | 100 | 10 | 1271.90 | 806.65 | 2.5 | 48019.23 | 31561.55 | 0.58 | 0.38 |
| 5C | | | | | | | | | |
| Example | 100 | 2 | 3152.39 | 1781.37 | 4.0 | 139090.05 | 80100.64 | 1.67 | 0.96 |
| 5D | | | | | | | | | |

| | | AUC_{inf} | | BA_{inf} | | | |
|---|---|---|---|---|---|---|---|
| | | (ng · h/mL) | | (%) | | | % |
| | CV | | Std | | Std | CV | Extrap- |
| | (%) | Mean | Dev | Mean | Dev | (%) | olated |
| 10 mg IV | 13.81 | 1198538.03 | 115842.07 | 100.00 | n/a | 9.67 | 20.06 |
| Control | | | | | | | |
| Example | 26.22 | 58433.71 | 18453.53 | 0.49 | 0.15 | 31.58 | 44.49 |
| 5A | | | | | | | |
| Example | 50.32 | 104519.28 | 55312.23 | 0.87 | 0.46 | 52.92 | 42.55 |
| 5B | | | | | | | |
| Example | 65.73 | 88109.15 | 58706.10 | 0.74 | 0.49 | 66.63 | 46.92 |
| 5C | | | | | | | |
| Example | 57.59 | 254136.78 | 153831.22 | 2.12 | 1.28 | 60.53 | 44.08 |
| 5D | | | | | | | |

### Example 6

### Proposed Formulations for First-in-Human (FIH) Studies

The proposed formulations for first-in-human (FIH) dosing are shown below in Table 13

TABLE 13
Projected Human Formulations

| Dose (mg) | 20 | 40 | 80 | 160 | 200 |
|---|---|---|---|---|---|
| Sodium | 660 | 660 | 660 | 660 | 660 |
| Decanoate (mg) | | | | | |
| Number of | 24 | 24 | 24 | 24 | 24 |
| Tablets | | | | | |
| Tablet Strength | 0.833 | 1.67 | 3.33 | 6.67 | 8.33 |
| (mg) | | | | | |
| Tablet Weight | 100 | 100 | 100 | 100 | 100 |
| (mg) | | | | | |

| | % | | | % | | | % | |
|---|---|---|---|---|---|---|---|---|
| Ingredient | w/w | mg/tab | mg/dose | w/w | mg/tab | mg/dose | w/w | mg/tab |
| Cmpd. formula | 0.833 | 0.833 | 20 | 1.67 | 1.67 | 40 | 3.33 | 3.33 |
| (I) | | | | | | | | |
| Sodium | 27.5 | 27.5 | 660 | 27.5 | 27.5 | 660 | 27.5 | 27.5 |
| Decanoate | | | | | | | | |
| Microcrystalline | 42.447 | 42.447 | 1,019 | 41.89 | 41.89 | 1,005 | 40.78 | 40.78 |
| Cellulose | | | | | | | | |
| Mannitol | 21.22 | 21.22 | 509 | 20.94 | 20.94 | 503 | 20.39 | 20.39 |
| Croscarmellose | 4 | 4 | 96 | 4 | 4 | 96 | 4 | 4 |
| sodium | | | | | | | | |
| Silicon dioxide | 2 | 2 | 48 | 2 | 2 | 48 | 2 | 2 |
| Magnesium | 2 | 2 | 48 | 2 | 2 | 48 | 2 | 2 |
| Stearate | | | | | | | | |
| Total | 100 | 100 | 2,400 | 100 | 100 | 2,400 | 100 | 100 |

| | | % | | | % | | |
|---|---|---|---|---|---|---|---|
| Ingredient | mg/dose | w/w | mg/tab | mg/dose | w/w | mg/tab | mg/dose |
| Cmpd. formula | 80 | 6.67 | 6.67 | 160 | 8.33 | 8.33 | 200 |
| (I) | | | | | | | |
| Sodium | 660 | 27.5 | 27.5 | 660 | 27.5 | 27.5 | 660 |
| Decanoate | | | | | | | |
| Microcrystalline | 979 | 38.55 | 38.55 | 925 | 37.45 | 37.45 | 925 |
| Cellulose | | | | | | | |
| Mannitol | 489 | 19.28 | 19.28 | 463 | 18.72 | 18.72 | 899 |
| Croscarmellose | 96 | 4 | 4 | 96 | 4 | 4 | 96 |
| sodium | | | | | | | |
| Silicon dioxide | 48 | 2 | 2 | 48 | 2 | 2 | 48 |
| Magnesium | 48 | 2 | 2 | 48 | 2 | 2 | 48 |
| Stearate | | | | | | | |
| Total | 2,400 | 100 | 100 | 2,400 | 100 | 100 | 2,400 |

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A pharmaceutical composition comprising:
a cyclic peptide; and
a decanoic acid salt.

2. The pharmaceutical composition of claim 1, wherein the decanoic acid salt is sodium decanoate.

3. The pharmaceutical composition of claim 1 or 2, wherein the cyclic peptide is Compound (I) having the formula: 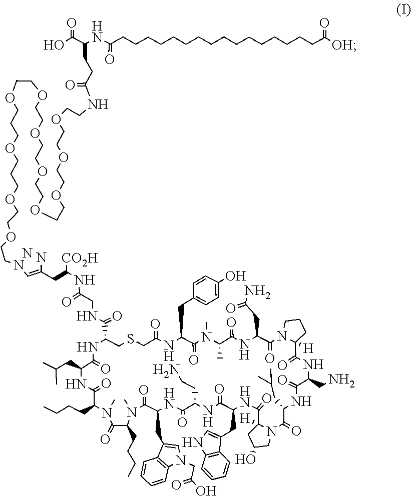 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]
or a pharmaceutically acceptable salt thereof.

4. The pharmaceutical composition of any one of claims 1 to 3, wherein the cyclic peptide is present in an amount of about 0.1 wt % to about 20 wt %.

5. The pharmaceutical composition of any one of claims 1 to 4, wherein the cyclic peptide is present in an amount of about 0.5 wt % to about 10 wt %.

6. The pharmaceutical composition of any one of claims 1 to 5, wherein the cyclic peptide is present in an amount of about 6.0 wt % to about 9.50 wt %.

7. The pharmaceutical composition of any one of claims 1 to 5, wherein the cyclic peptide is present in an amount of about 0.8 wt % to about 5.0 wt %.

8. The pharmaceutical composition of any one of claims 2 to 7, wherein the sodium decanoate is present in an amount of about 15 wt % to about 70 wt %.

9. The pharmaceutical composition of any one of claims 2 to 8, wherein the sodium decanoate is present in an amount of about 20 wt % to about 55 wt %.

10. The pharmaceutical composition of any one of claims 2 to 9, wherein the sodium decanoate is present in an amount of about 20 wt % to about 30 wt %.

11. The pharmaceutical composition of any one of claims 2 to 10, wherein the sodium decanoate is present in an amount of about 27 wt % to about 28 wt %.

12. The pharmaceutical composition of any one of claims 1 to 11, wherein the composition is for oral administration.

13. The pharmaceutical composition of any one of claims 1 to 12, wherein the composition comprises a capsule, tablet, minitablet, or sachet.

14. A pharmaceutical composition comprising:
a. Compound (I) having the formula: 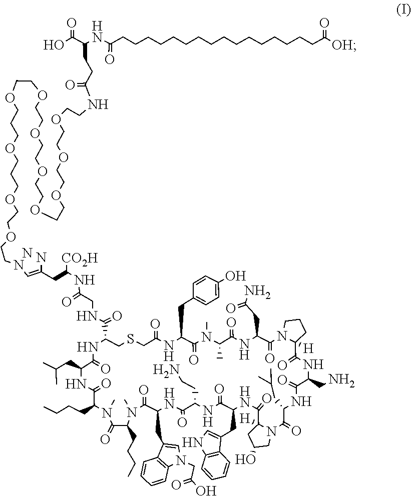 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]
or a pharmaceutically acceptable salt thereof;
b. about 27.5 wt % sodium decanoate;
c. about 37 wt % to about 43 wt % microcrystalline cellulose;
d. about 18 wt % to about 22 wt % mannitol;
e. about 4 wt % croscarmellose sodium;
f. about 2 wt % silicon dioxide; and
g. about 2 wt % magnesium stearate.

15. The pharmaceutical composition of claim 14, wherein Compound (I), or the pharmaceutically acceptable salt thereof, is present in an amount of about 0.8%.

16. The pharmaceutical composition of claim 14, wherein Compound (I), or the pharmaceutically acceptable salt thereof, is present in an amount of about 1.7%.

17. The pharmaceutical composition of claim 14, wherein Compound (I), or the pharmaceutically acceptable salt thereof, is present in an amount of about 3.3%.

18. The pharmaceutical composition of claim 14, wherein Compound (I), or the pharmaceutically acceptable salt thereof, is present in an amount of about 6.7%.

19. The pharmaceutical composition of claim 14, wherein Compound (I), or the pharmaceutically acceptable salt thereof, is present in an amount of about 8.3%.

20. The pharmaceutical composition of any one of claims 14 to 19, wherein the composition is for oral administration.

21. The pharmaceutical composition of any one of claims 14 to 20, wherein the composition comprises a capsule, tablet, minitablet, or sachet.

22. The pharmaceutical composition of any one of claims 2 to 21, wherein the sodium decanoate is crystallized.

23. A method of improving bioavailability of a cyclic peptide in a subject in need thereof comprising formulating the cyclic peptide with sodium decanoate.

24. The method of claim 23, wherein the bioavailability is improved at least by about 0.1%, at least by about 0.2%, at least by about 0.3%, at least by about 0.4%, at least by about 0.5%, at least by about 0.6%, at least by about 0.7%, at least by about 0.8%, at least by about 0.9%, or at least about 1.0%.

25. The method of claim 23 or 24, wherein the cyclic peptide is Compound (I): 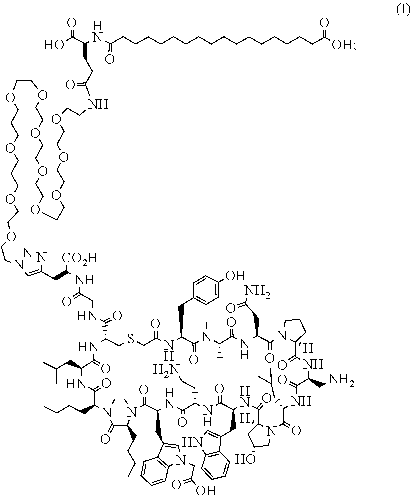 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]
or a pharmaceutically acceptable salt thereof.

26. The method of any one of claims 23 to 25, wherein the sodium decanoate is crystallized.

27. A method of inhibiting growth, proliferation, or metastasis of cancer cells in a subject in need thereof, said method comprising administering to the subject a therapeutically effective amount of a pharmaceutical composition of any one of claims 1 to 22.

28. The method of claim 26, wherein the cancer is selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and a hematological malignancy.

29. A method of enhancing, stimulating, and/or increasing an immune response in a subject in need thereof, said method comprising administering to the subject a therapeutically effective amount of a pharmaceutical composition of any one of claims 1 to 22.
